(19) United States
(12) Patent Application Publication
(10) Pub. No.: US 2007/0264368 A1
(43) Pub. Date: Nov. 15, 2007

(54) Carbohydrase inhibitors derived from fagaceous plants and use thereof

(76) Inventor: Takahiro Tsujita, (US)

Correspondence Address: FITCH, EVEN, TABIN & FLANNERY, P. O. BOX 18415, WASHINGTON, DC, 20036, US

(21) Appl. No.: 11/662,636

(22) PCT Filed: Jun. 3, 2005
(86) PCT No.: PCT/JP05/10620

§ 371 Date: Mar. 13, 2007

(30) Foreign Application Priority Data
Sep. 13, 2004 (JP) 2004-265281
Dec. 28, 2004 (JP) 2004-381581

(51) Int. Cl. A61K 36/49

(52) U.S. Cl. 424/769; 424/771

(57) ABSTRACT

The present invention provides a plant-derived carbohydrase inhibitor, wherein the inhibitor is effective for preventing or alleviating diabetes, or preventing obesity, and foods, drinks, and medicines containing the same. The present invention is accomplished by use of an α-amylase inhibitor or an α-glucosidase inhibitor as a carbohydrase inhibitor that is extracted from an entire or a part of fagaceous plants using water, another polar solvent, or a mixture thereof. The present invention can also be accomplished by adding the carbohydrase inhibitor to a food or medical composition as an active ingredient for delaying saccharide digestion or absorption, suppressing a rise in postprandial blood glucose levels, or preventing obesity.

### TECHNICAL FIELD

The present invention relates to carbohydrase inhibitors derived from fagaceous plants, and in particular of inhibitors to α-amylase or α-glucosidase. Furthermore, the present invention relates to various uses of the carbohydrase inhibitors, and specifically to medical compositions and food compositions which utilize the physiological actions of the inhibitors.

### BACKGROUND ART

The prevalence of diabetes is increasing due to changes in modern lifestyles. In Japan, it is estimated that the number of diabetic patients, including potential diabetic patients, is more than 15 million. Most of the diabetic patients suffer from type II diabetes. Type II diabetes mellitus is closely related to obesity, and causes chronic hyperglycemia due to insulin resistance, etc. Furthermore, type II diabetes causes complications, such as retinopathy, nephritis, and neurological disorders. Diet and exercise therapy are the key factors for preventing and treating type II diabetes. In dieting, controlling blood glucose levels in everyday life is especially important. Blood glucose levels are greatly affected by the saccharides (starches, glycogen, sugars, etc.) contained in food. These saccharides are decomposed by the actions of α-amylase and α-glucosidase, which are digestive enzymes (carbohydrases). α-Amylase is an endo-type enzyme that hydrolyzes the α-1,4-glucoside linkages of starches and glycogen. These enzymes are contained in the saliva and pancreatic fluid of animals, and transform starches and the like into maltose, etc., in the alimentary canal.

Disaccharides, such as maltose and sucrose, are transformed into glucose by hydrolysis due to α-glucosidase, which exists in the cell membrane of the small intestine mucous membrane, and are absorbed. Maltase, which decomposes maltose, and sucrase, which decomposes sucrose, are typical α-glucosidases. Glucose absorbed from the small intestine is carried into the blood, and raises the blood glucose level. Therefore, to inhibit a superfluous energy supply or control blood glucose levels, in other words, to prevent or treat obesity and diabetes, it is very important to control the activity of these enzymes such as α-amylase and α-glucosidase.

Much research has been conducted on substances that inhibit the action of carbohydrases and many carbohydrase inhibitors have been discovered. Examples include a protein-based substance that is derived from wheat [O'Donnell M D and McGeeney K F.: Purification and properties of an alpha-amylase inhibitor from wheat. Biochim. Biophys. Acta, 422, 159-169 (1976)]; a polysaccharide extracted from soybean (Japanese Unexamined Patent Publication No. 1991-290187; protein-based substances NSA1-I and NSA1-II extracted from cocoyam (Colocasia esculenta) (Japanese Unexamined Patent Publication No. 1991-294300; crude extracts from laurel (Laurus nobillis L) (Japanese Unexamined Patent Publication No. 1992-27389); extracts from guava leaves (Japanese Unexamined Patent Publication No. 1995-59539); banaba (Laqerstroemia speciosa) extracts obtained by using hot water [Hosoyama H, Sugimoto A, Suzuki Y, et al.: Isolation and Quantitative Analysis of the alpha-amylase Inhibitor in Lagerstroemia speciosa (L.) Pers. (Banaba) J. Pharm. Soc. Jpn., 123, 599-605, (2003)]; extracts from the above-the-ground root of ephedra (Ephedra Herb) (Japanese Unexamined Patent Publication No. 1997-2963); extracts from black rice (Japanese Unexamined Patent Publication No. 2004-91462); etc. Moreover, an oligosaccharide produced from actinomyces is known as a carbohydrase inhibitor derived from microorganisms.

Furthermore, examples of well known commercially available medicines (anti-diabetic medicines) having inhibition activities against α-glucosidase or α-amylase include acarbose (Glucobay; manufactured by Bayer Yakuhin, Ltd.) [Jenkins D J, Taylor R H, Nineham R. et al.: “Combined use of guar and acarbose in reduction of postprandial glycaemia”, Lancet 2(8149) 924-927 (1979)] and voglibose (Basen; manufactured by Takeda Pharmaceutical Company Limited)[Yoshio Goto, Shigeaki Baba, Masakazu Nakagawa, et al.: “Effectiveness of AO-128, an α-glucosidase inhibitor, for treating non-insulin dependent diabetes mellitus.” IGAKU NO AYUMI 160 943-971 (1992)]. Furthermore, the anti-obesity action of carbohydrase inhibitors is disclosed in a document written by Svensson, et al., [Svensson B, Fukuoka K, Nielsen P K, et al.: “Proteinaceous amylase inhibitors”, Biochim. Biophys. Acta, 1696, 145-156 (2003)] and one written by Udani, et al., [Udani J, Hardy M and Madsen D C: “Blocking carbohydrate absorption and weight loss: A clinical trial using Phase 2 brand proprietary fractionated white bean extract”, Alternative Medicine Review 9, 63-69 (2003)].

As described above, many α-amylase inhibitors and α-glucosidase inhibitors have been proposed. However, in order to put these substances into practical use as effective agents for preventing or treating diabetes or obesity, in addition to the strength of the inhibitory activity against α-amylase or α-glucosidase, studies are necessary from many viewpoints, such as safety and the existence of side effects in the body, and the availability of a stable supply of raw materials, etc. Given this, known inhibitors are not necessarily satisfactory.

### DISCLOSURE OF THE INVENTION

An object of the present invention is to provide a carbohydrase inhibitor obtained from plants, and specifically a carbohydrase inhibitor that exhibits an inhibitory activity against α-amylase or α-glucosidase. Another object of the present invention is to provide a carbohydrase inhibitor that is safe to the body and prepared from a material that can be supplied stably.

The carbohydrase inhibitor can delay the digestion and absorption of saccharides from the alimentary canal. This makes it possible to reduce a rise in blood glucose levels after a meal. Furthermore, because the carbohydrase inhibitor can delay the digestion and absorption of saccharides from the alimentary canal, it is expected to have an anti-obesity effect. Therefore, the present invention provides medical compositions and food compositions using these physiological actions of the carbohydrase inhibitor.

In order to find novel carbohydrase inhibitors, the present inventors conducted screening using materials which are generally disposed in daily life, such as juice extraction lees from citrus fruits, juice extraction lees from aojiru (juice from a green, leafy vegetable), peels of various kinds of fruit, bittern, chitin and chitosan of crustacea, and testa or gut of fish. As a result, they found that solvent extracts from the seeds (astringent skins and shells), leaves, testa, etc., of agaceous plants have a strong α-amylase inhibitory activity or α-glucosidase inhibitory activity. Furthermore, they found that these solvent extracts could reduce a rise in blood glucose level for normal and diabetic rats, and humans after meals (or after eating saccharides). The present invention was accomplished based on these findings. In other words, the present invention includes the following features:

Item 1. A carbohydrase inhibitor comprising fagaceous plant solvent extract.

Item 2. The carbohydrase inhibitor according to Item 1, which is obtained by subjecting an entire or part of a fagaceous plant to extraction using water, organic solvent, or a mixture thereof.

Item 3. The carbohydrase inhibitor according to Item 1, wherein the fagaceous plant belongs to the Castanea genus, Castanopsis genus, or Quercus genus, and the carbohydrase inhibitor can be obtained by subjecting the shell, astringent skin, case, leaf, bark, seed (nut and cotyledon) thereof, or a portion containing at least one of these, to extraction using water, organic solvent, or a mixture thereof.

Item 4. The carbohydrase inhibitor according to any one of Items 1 to 3, which can be obtained by subjecting the shell, astringent skin, case, leaf, bark or seed (nut and cotyledon) of at least one plant selected from the group consisting of Castanea crenata, Quercus acutissima, and Castanopsis cuspidata to extraction using water, organic solvent, or a mixture thereof.

Item 5. The carbohydrase inhibitor according to any one of Items 1 to 4, wherein the carbohydrase to be inhibited is α-amylase, α-glucosidase, or both.

Item 6. A composition for delaying saccharide digestion and absorption, the composition comprising a carbohydrase inhibitor according to any one of Items 1 to 5 as an active ingredient.

Item 7. A composition for reducing a postprandial rise in blood glucose levels comprising, as an active ingredient, a carbohydrase inhibitor of any one of Items 1 to 5.

Item 8. A composition for ameliorating hyperglycemia comprising, as an active ingredient, a carbohydrase inhibitor of any one of Items 1 to 5.

Item 9. An anti-obesity composition comprising, as an active ingredient, a carbohydrase inhibitor of any one of Items 1 to 5.

Item 10. A food composition comprising a carbohydrase inhibitor of any one of Items 1 to 5.

Item 11. The food composition according to Item 10, which is a beverage or a food high in carbohydrates, such as a noodle, a bread or confectioneries.

Item 12. A food composition comprising an effective amount of a carbohydrase inhibitor according to any one of Items 1 to 5 as an active ingredient for delaying saccharide digestion and absorption.

Item 13. The food composition according to Item 12, which has the property of delaying saccharide digestion and absorption, and whose package has a note stating that the food composition is suitable for delaying digestion and absorption of saccharides.

Item 14. A food composition comprising an effective amount of a carbohydrase inhibitor according to any one of Items 1 to 5 as an active ingredient for reducing a postprandial rise in blood glucose levels or for ameliorating hyperglycemia.

Item 15. The food composition according to Item 14, which has the property of suppressing postprandial rise in blood glucose levels or for ameliorating hyperglycemia, and whose package has a note that the food composition is suitable for suppressing a postprandial rise in blood glucose levels or for ameliorating hyperglycemia.

Item 16. A food composition comprising an effective amount of carbohydrase inhibitor according to any one of Items 1 to 5 as an active ingredient for preventing obesity.

Item 17. The food composition according to Item 17, which has an anti-obesity effect, and whose package has a note stating that the food composition is suitable for preventing obesity.

Item 18. A medical composition comprising, as an active ingredient, a carbohydrase inhibitor according to any one of Items 1 to 5.

Item 19. The medical composition according to Item 18, which is a medicine for preventing or treating diabetes.

Item 20. The medical composition according to Item 18, which is an anti-obesity medicine.

Item 21. A method for suppressing a postprandial rise in blood glucose levels or ameliorating hyperglycemia of a subject, comprising injecting or otherwise administering a carbohydrase inhibitor according to any one of Items 1 to 5 to the subject.

Item 22. A method for preventing or reducing obesity comprising injecting or otherwise administering a carbohydrase inhibitor according to any one of Items 1 to 5 to the subject.

Item 23. Use of a carbohydrase inhibitor according to any one of Items 1 to 5, for producing a composition for delaying saccharide digestion and absorption.

Item 24. Use of a carbohydrase inhibitor according to any one of Items 1 to 5, for producing a composition for suppressing a postprandial rise in blood glucose levels.

Item 25. Use of a carbohydrase inhibitor according to any one of Items 1 to 5, for producing a composition for ameliorating hyperglycemia.

Item 26. Use of a carbohydrase inhibitor according to any one of Items 1 to 5, for producing an anti-obesity composition.

### BRIEF DESCRIPTION OF DRAWINGS

FIG. 1 shows the effect of chestnut astringent skin extract (-◯-), chestnut shell extract (-●-), chestnut leaf extract (-□-), and hot water extract from guava leaf (-▪-) on α-amylase activity (%) (Experiment 1).

FIG. 2 shows the effect of chestnut astringent skin extract (-◯-), chestnut shell extract (-●-), chestnut leaf extract (-□-), and hot water extract from guava leaf (-▪-) on α-glucosidase (maltase) activity (%) (Experiment 2(2)).

FIG. 3 shows the effect of chestnut astringent skin extract (-◯-), chestnut shell extract (-●-), chestnut leaf extract (-□-), and hot water extract from guava leaf (-▪-) on α-glucosidase (sucrase) activity (%) (Experiment 2(3)).

FIG. 4 shows the change in the blood glucose levels after administering chestnut astringent skin extract (10 mg/kg of body weight: -▴-, 25 mg/kg of body weight: -Δ-, 50 mg/kg of body weight: -▪-, 100 mg/kg of body weight: -□-, 300 mg/kg of body weight) and starch (2 g/kg body weight) to normal rats (-●-) (Experiment 3). As a control, the change in the blood glucose level of normal rats that was administered with only starch (2 g/kg of body weight) is also shown (-◯-).

FIG. 5 shows the change in the blood glucose of diabetic rats administered with chestnut astringent skin extract (300 mg/kg of body weight) and starch (2 g/kg of body weight) (-●-) (Experiment 4). As a control, the change in the blood glucose levels of diabetic rats administered with only starch (2 g/kg of body weight) is also shown (-◯-).

FIG. 6 shows the change in the blood glucose of human subjects administered with chestnut astringent skin extract (2 g) while consuming boiled rice (200 g) (-●-) (Experiment 5). As a control, the change in the blood glucose levels of human subjects administered water while consuming boiled rice (200 g) is also shown (-◯-).

### BEST MODE FOR CARRYING OUT THE INVENTION

(1) Carbohydrase Inhibitor

A carbohydrase inhibitor of the present invention can be obtained by extracting from an entire or part of a fagaceous plant with a solvent.

Such fagaceous plants are not limited and examples thereof include chestnut (Castanea crenata) belonging to the Castanea genus; Japanese chinquapin (Castanopsis cuspidata) and Suda-jii (Castanopsis sieboldii) belonging to the Castanopsis genus; Japanese beech (Fagus crenata), Japanese blue beech (Fagus japonica), Japanese Stone oak (Lithocarpus edulis), and Japanese oak (Lithocarpus glabra) belonging to the Fagus genus; Oriental white oak (Quercus aliena), Sawtooth oak (Quercus acutissima), Mizunara oak (Quercus crispula), Japanese emperor oak (Quercus dentata), Konara oak (Quercus serrata), Chinese cork oak (Quercus variabilis), Japanese evergreen oak (Quercus acuta), Arakashi (Quercus glauca), Japanese white oak (Quercus myrsinaefolia), Umeba-gashi (Quercus phillyraeoides), Urajiro-gashi (Quercus salicina), and Tsukubane-gashi (Quercus sessilifolia) belonging to the Quercus genus, etc. Preferable examples are plants that belong to the Castanea genus, Castanopsis genus, or Quercus genus. More preferable examples are chestnuts of the Castanea genus, Suda-jii of the Castanopsis genus, and Sawtooth oak that of the Quercus genus.

As a raw material for the carbohydrase inhibitor of the present invention, it is possible to use an entire fagaceous plant, or a part thereof such as the bark, a root, bur (spines, flesh), fruit, testa (shell and astringent skin), leaf, seed (nut and cotyledon), or petal. Preferable examples are plant parts, such as the bark, a leaf, bur (spines, flesh), and testa (shell and astringent skin), and portions including at least one of these, and especially preferable examples are plant parts, such as shell and astringent skin, and portions including at least one of these.

The bark and astringent skin of a chestnut include tannins, gallic acid, flavonoids, etc., and are believed to be effective in treating heat rash and skin burn because of an antiphlogistic effect. They are also believed to be effective in preventing lifestyle-related diseases, such as arteriosclerosis, by improving blood circulation and preventing deposition of cholesterol in the blood vessels. However, there is no report stating that they inhibit the actions of carbohydrases, such as α-amylase and α-glucosidase, nor that they suppress a rise in blood glucose levels by means of this inhibitory effect.

The form of the fagaceous plant (whole or part) to be subjected to extraction is not limited and may be raw or dried and the plant may be crushed or pulverized into a desired shape such as chips or powder.

The solvent used for extraction is not limited, and, water, organic solvents or a mixture thereof can be used. Preferable organic solvent is lower alcohols, polyhydric alcohols, and like polar organic solvent. Specifically, examples of lower alcohols are methanol, ethanol, propanol, isopropanol, butanol and like C₁-C₆ alcohols, and more preferably, C₁-C₄ alcohols. Examples of polyhydric alcohols are glycerol, 1,3-butylene glycol, propylene glycol, dipropylene glycol, polyethylene glycol, etc. Examples of polar organic solvents other than those mentioned above are acetone, methyl ketone, ethyl ketone and like ketones; ethyl acetate, methyl acetate, butyl acetate and like esters; ethyl ether, propyl ether and like ethers; acetonitrile; etc.

Such solvents may be used singly or as a combination of two or more. Examples of combinations of two or more solvents are those combining water with a lower alcohol, polyhydric alcohol, or other polar organic solvent.

Preferable examples thereof include a mixture of water, acetone and acetonitrile, a mixture of water and acetone (aqueous acetone solution, water-containing acetone), and a mixture of water and acetonitrile (aqueous acetonitrile solution, water-containing acetonitrile,). When a mixture of water and another polar solvent (preferably, a water-containing acetone or aqueous acetonitrile solution) is used as an extractant, the content of the organic solvent is not limited, and is for example usually 10-90 vol. %, and preferably 40-60 vol. %.

Generally used methods can be employed in the extraction method. There is no restriction on the extraction method, and examples thereof include a method wherein an entire or part of the above-mentioned plant (raw, dried, crushed, or pulverized), is subjected to cold extraction, hot extraction, or dipped in a solvent while heating; a percolation method, etc. The extraction temperature is not particularly limited and can be suitably selected from the range of 4 to 100° C. Usually, extraction can be conducted at room temperature. Dipping of the plant may be conducted while the solvent is allowed to stand, or while stirring or shaking. The extraction time is not particularly limited and can be suitably selected from the range of 1 hour to 2 weeks. Usually, the extraction time is about 5 hours. The volume of the extractant is also not limited. It is preferable that extraction be repeated 2 to 3 times using a solvent in an amount 10 to 30 times (weight ratio) that of the plant being extracted on a dry-weight basis.

It is also possible to conduct extraction using a solvent in a supercritical or subcritical state (supercritical extraction method or subcritical extraction method). In supercritical and subcritical extraction methods, extraction is conducted using a solvent in a supercritical or subcritical state (in a state that both the temperature and pressure of the solvent exceed critical values, in other words, the solvent is in an intermediate state between liquid and gas). Examples of extractants are carbon dioxide, ethylene, ethane, propane, water, etc.; however, from the viewpoint of safety and non-toxicity, etc., carbon dioxide is preferable.

The extraction pressure and temperature are not limited as long as the extractant becomes supercritical or subcritical, and can be suitably selected depending on the extractant to be used. Specifically, the extraction pressure can be selected from a range of 3-70 MPa, and, for example, when carbon dioxide is used as an extractant, it is preferable to select the extraction pressure from a range of 5-40 MPa. The extraction temperature can usually be selected from the range of 25-200° C., and preferably 25-100° C. There is no restriction; however, it is possible to use an entrainer to enhance the solubility of the extractant. Examples of entrainers include water; methanol, ethanol, and like C₁-C₄ lower alcohols; acetone, acetonitrile, etc. When an entrainer is used, it is usually preferable that the content of the entrainer in the extractant be 0.00001-50.0 wt %, and more preferably 0.0001-10 wt %. The extraction time is not limited and can be suitably selected from the range of 2 hours to 2 weeks.

If necessary, solid substances are removed from the obtained extract by filtration, centrifugation and/or other solid-liquid separation methods. Depending on the mode of use, the extract may be used as is, or partially concentrated or dried by evaporating the solvent, and the extract can be used as a plant essence or dried plant essence. The thus obtained fagaceous plant solvent extract, preferably a solvent extract from a plant belonging to the Castanea genus, Castanopsis genus or Quercus genus (preferably, a solvent extract from the shell, astringent skin, bark or leaves thereof) has an inhibitory activity against carbohydrases, such as α-amylase and α-glucosidase as described in the Examples below. Therefore, these solvent extracts can be used for foods, medicines, feeds, reagents, etc., as an ingredient, for inhibiting the activities of α-amylase, α-glucosidase and like carbohydrases.

The above-described plant essence or dried plant essence may be purified by washing it with a solvent in which the plant essence is insoluble. It is also possible to use the plant essence or dried plant essence by dissolving or suspending it into an additional suitable solvent.

The above-described plant essence or dried plant essence may be highly purified using a known purifying method, and the thus obtained purified substance may be used as a carbohydrase inhibitor. There is no restriction on the purification method, and an example thereof is a countercurrent distribution method, chromatography, etc., wherein carbohydrase inhibition activities (e.g., α-amylase inhibitory activity and α-glucosidase inhibitory activity) are measured and a fraction having at least one of these activities is selected. Several methods for measuring α-amylase inhibitory activity and α-glucosidase inhibitory activity are known, and any such method can be employed. Specifically, purification can be conducted by following the method as described later in the Examples. The purified extract obtained by employing any of various purification methods may be dried by decompression drying, freeze-drying or the like standard drying method and can be used as a carbohydrase inhibitor.

(2) Use of Carbohydrase Inhibitor

The above-described carbohydrase inhibitor of the present invention can be used as a reagent (chemical product) as an α-amylase inhibitor or α-glucosidase inhibitor due to its α-amylase inhibitory activity or α-glucosidase inhibitory activity.

A carbohydrase inhibitor of the present invention has the property of delaying digestion and absorption of saccharides in vivo (intestinal) and suppressing a postprandial rise of blood glucose levels (hyperglycemia) due to its α-amylase inhibitory activity or α-glucosidase inhibitory activity. Therefore, the carbohydrase inhibitor of the present invention can be used as an active ingredient of a composition for delaying the digestion and absorption of saccharides (a digestion and absorption retardant), a composition for suppressing a postprandial rise in blood glucose levels (an inhibitor of rising blood glucose levels), or a composition for ameliorating hyperglycemia (hyperglycemia improver).

(3) A composition for delaying digestion and absorption of saccharides, a composition for suppressing a postprandial rise in blood glucose levels, a composition for ameliorating hyperglycemia, and an anti-diabetic composition.

As described above, the present invention provides a composition for delaying digestion and absorption of saccharides, a composition for suppressing a postprandial rise in blood glucose levels, and a composition for ameliorating hyperglycemia.

The composition for delaying the digestion and absorption of saccharides may contain the above-described carbohydrase inhibitor in an amount that is effective for delaying the digestion and absorption of saccharides in the alimentary canal. A composition for suppressing a postprandial rise in blood glucose levels or the composition for ameliorating hyperglycemia may contain the above-described carbohydrase inhibitor in an amount that is effective for suppressing the postprandial rise in blood glucose levels. Usually, a composition for delaying the digestion and absorption of saccharides, composition for suppressing a postprandial rise in blood glucose levels or a composition for ameliorating hyperglycemia should contain 0.1-100 parts by weight of carbohydrase inhibitor of the present invention per 100 parts by weight of the total composition. A composition for delaying the digestion and absorption of saccharides, a composition for suppressing a postprandial rise in the blood glucose level, or a composition for ameliorating hyperglycemia may comprise, in addition to the above-described carbohydrase inhibitor, pharmaceutically acceptable carriers and additives and/or carriers and additives that are permitted to be added to foods.

The carbohydrase inhibitor of the present invention can be used as an active ingredient of an anti-obesity composition (anti-obesity agent) that prevents obesity caused by hyperphagia, since the carbohydrase inhibitor has the property of inhibiting digestion of saccharides, such as starches and sugars contained in foods, and prevents them from being absorbed as energy. Therefore, the present invention provides an anti-obesity composition comprising the carbohydrase inhibitor as an active ingredient. The composition may contain the carbohydrase inhibitor in an amount that is effective for resolving or suppressing obesity. Usually, the anti-obesity composition contains 0.1-100 parts by weight of the carbohydrase inhibitor of the present invention per 100 parts by weight of the total composition. The anti-obesity composition may comprise, in addition to the above-described carbohydrase inhibitor, pharmaceutically acceptable carriers and/or additives or carriers and additives that are permitted to be added to foods.

(4) Food Composition and Medical Composition

As an example of a more specific and practical mode, the carbohydrase inhibitor of the present invention can be used as an active ingredient of a food or medical composition, and prepared into food or medicine. Due to its α-amylase inhibitory activity or α-glucosidase inhibitory activity, such a food or medical composition of the present invention has the property of delaying digestion and absorption of saccharides in vivo (intestinal), suppressing a postprandial rise of blood glucose levels, ameliorating hyperglycemia, and/or preventing obesity.

Therefore, the present invention provides a food or medical composition that has the above-described effects by comprising the carbohydrase inhibitor. Such food or medical compositions are not limited to only those for humans but also include those for various animals, in particular, other mammals. Therefore, the food compositions include foods for animals such as cats, dogs, and the like pets, and the medical compositions include those for animals other than humans.

(4-1) Food Composition

Since the food composition of the present invention has a property of delaying the digestion and absorption of saccharides, suppressing a rise in postprandial blood glucose levels, and/or ameliorating hyperglycemia as described above, the food composition of the present invention has an effect for preventing diabetes and/or the progress thereof, or preventing diseases caused by postprandial hyperglycemia. Therefore, the food composition of the present invention is useful as a health food or a functional food for a subject (including human subjects) suffering from a relatively high blood glucose level or a subject (including human subjects) whose blood glucose level may be of concern. Such a food composition is not limited as long as it comprises the carbohydrase inhibitor in an amount effective for delaying the digestion and absorption of saccharides in the alimentary canal, suppressing a rise in postprandial blood glucose levels, or ameliorating hyperglycemia. If necessary, the composition may contain carriers and/or other additives permitted to be added to foods.

Since the food composition of the present invention has an effect for delaying the digestion and absorption of saccharides in vivo (intestinal), it is possible to provide a food composition of the present invention as a so-called anti-obesity food, i.e., the subject will not easily gain weight through eating the food. The food composition is not limited as long as it comprises the carbohydrase inhibitor in an amount effective for delaying the digestion and absorption of saccharides in the alimentary canal, and, if necessary, the composition may contain carriers and/or other additives permitted to be added to foods.

The forms of such food compositions are not limited, and the carbohydrase inhibitor may be prepared into supplements (functional foods) having the form of tablets, pills, capsules, granules, pulvis, powders, troches, solutions (drinks), etc., together with carriers and/or other additives permitted to be added to foods, if necessary.

The food compositions of the present invention include foods (for example, foods for specified health use, dietary supplements, functional foods, etc.) that have various effects due to their α-amylase inhibitory activity or α-glucosidase inhibitory activity by comprising the carbohydrase inhibitor. The foods for specified health use encompassed by the present invention include the foods which have a property of, by containing the carbohydrase inhibitor, delaying the digestion and absorption of saccharides, suppressing a rise in postprandial blood glucose levels, and/or ameliorating hyperglycemia, and therefore such foods have labels on their packages stating that they are useful for delaying digestion and absorption of saccharides, suppressing a rise in postprandial blood glucose levels (hyperglycemia), and/or ameliorating hyperglycemia. There is no specific limitation on the expressions of the labels and include, for example, “suitable for those who care about blood glucose levels”, “suitable for those who have relatively high blood glucose level”, or “moderating absorption of saccharides”.

The foods for specified health use encompassed by the present invention include those comprising the carbohydrase inhibitor and having an effect for delaying digestion and absorption of saccharides, and therefore such foods have labels on their packages stating that they are useful for reducing or preventing obesity (i.e., loosing weight). The expressions on the package is not limited, and examples thereof include “suitable for those who are concerned about their weight”, “suitable for those who are over-weight”, etc.

Examples of such foods include milk-based beverages, lactobacillus beverages, fruit juice-containing beverages, soft drinks, carbonated beverages, fruit juices, vegetable juices, vegetable and fruit juice-containing beverages, alcoholic beverages, powdered beverages, coffee, black tea, green tea, barley tea, and like beverages; custard puddings, milk puddings, soufflé puddings, fruit juice-containing puddings and like puddings; jelly, bavarois, yogurt, and the like desserts; ice cream, iced milks, lact-ice, milk ice-cream, fruit juice-containing ice cream, soft ice cream, popsicles, sorbet, iced confectionery, and like cold sweets; chewing gum, bubble gum, and like gums (stick gums, sugar-coated grain gums, etc.); chocolates, such as marble-chocolate and like coated chocolates, as well as strawberry chocolate, blueberry chocolate, melon chocolate and like flavor-added chocolates; hard candies (including bonbons, butterballs, marbles, etc.), soft candies (including caramel, nougat, gummy candy, marshmallows, etc.), drops, toffee, and like caramels; cakes, hard biscuits, cookies, okaki (rice cracker), senbei (rice cracker), and like baked confections (these are referred to as confectioneries); breads; consommé soups, potages and like soups; miso, soy sauce, dressings, ketchup, sauces, furikake (seasoned powder for sprinkling over rice) and like seasonings; strawberry jam, blueberry jam, marmalade, apple jam, apricot jam, preserves and like jams; red wine and like fruit-based liquors; compote of cherries, apricots, apples, strawberries, peaches and like processed fruits; hams, sausages, roast pork and like processed meats; fish ham, fish sausage, fish surimi (pasted fish meat), kamaboko (pureed fish loaf), chikuwa (pureed and steamed fish cake), hanpen (pounded fish cake), satsumaage (deep fried fish paste), datemaki (sweet omelet with fish past), whale-bacon and like “fish” cakes; udon (thick white wheat noodles), hiyamugi (thin udon), somen (fine white wheat noodles), buckwheat noodles, Chinese noodles, spaghetti, macaroni, bifun (rice vermicelli), harusame (thin potato starch noodles), wonton and like noodles; and like various processed foods. Preferable examples are beverages, and foods high in carbohydrates, such as noodles, breads, and confectioneries.

The amount of the carbohydrase inhibitor contained in the above-mentioned food composition and the amount of the carbohydrase inhibitor intaken are not limited, and can be suitably selected from a wide range depending on the kind of food compositions, targeted functions and effects, and other conditions. The amount intaken thereof varies depending on the types of the food composition; however, the amount of carbohydrase inhibitor (for example, based on the dry weight of astringent skin of Castanea on a dry weight) taken per instance by a person whose body weight is 60 kg can be suitably selected form the range of about 10 to 200,000 mg/(60 kg body weight).

(4-2) Medical Composition

The medical composition of the present invention comprising a carbohydrase inhibitor as an active ingredient can be effectively used as an anti-diabetic medicine due to its effect of suppressing a postprandial rise in blood glucose levels (hyperglycemia) by delaying digestion and absorption of saccharides in vivo (intestinal).

The anti-diabetic medicine broadly encompasses those that can prevent or improve diabetes. Specifically, the anti-diabetic medicine of the present invention includes those that can prevent an onset of diabetes in a subject (including humans and other animals) who has the potential of suffering from an onset of diabetes, due to its effect for suppressing a postprandial rise in blood glucose levels. Furthermore, the anti-diabetic medicine of the present invention encompasses those that have an effect for ameliorating the hyperglycemic condition of a subject (including humans and other animals). The anti-diabetic medicine of the present invention also encompasses those that have an effect for preventing or ameliorating diseases attributable to hyperglycemia, such as diabetic complications, by suppressing or ameliorating blood glucose levels (reducing the blood glucose level from hyperglycemic condition). Note that the diabetes targeted by the present invention is preferably insulin-independent type II diabetes.

Diabetic complications are general or local diseases directly or indirectly caused by diabetes. Specific examples thereof are diabetic acidosis, diabetic xanthoma, diabetic myatrophy, diabetic ketosis, diabetic coma, diabetic stomach disorders, diabetic gangrene, diabetic ulcer, diabetic diarrhea, diabetic microangiopathy, diabetic uterosclerosis, diabetic cardiomyopathy, diabetic neuropathy, diabetic nephropathy, diabetic blister, diabetic cataract, diabetic dermatitis, diabetic scleredema, diabetic retinopathy, diabetic necrobiosis lipoidica, diabetic blood flow obstructions, etc.

The above-described carbohydrase inhibitor can be used as an anti-diabetic medicine (medical composition) as is. However, it is preferable that the carbohydrase inhibitor be used as an anti-diabetic medicine (medical composition) comprising the carbohydrase inhibitor in an amount effective for suppressing a rise in blood glucose levels together with pharmaceutically acceptable carriers and/or additives.

The medical composition of the present invention comprising a carbohydrase inhibitor as an active ingredient can be effectively used as an anti-obesity medicine due to its effect for delaying digestion and absorption of saccharides in vivo (intestinal). The above-described carbohydrase inhibitor can be used as an anti-obesity medicine (medical composition) by itself; however, it preferable that the carbohydrase inhibitor be used as an anti-obesity medicine (medical composition) comprising an effective amount of carbohydrase inhibitor for preventing obesity, and pharmaceutically acceptable carriers and/or additives.

The form of administration of the medical composition (form of a pharmaceutical preparation) can be suitably selected depending on the administration route. Medical compositions are generally classified into the following groups: orally administered medicines, nasally administered medicines, vaginally administered medicines, suppositories, sublingual tablets, non-orally administered medicines (injection or drops), etc. In the present invention, it is preferable that the composition be administered orally. By following a standard method, the composition of the present invention can be formed, or prepared into solid pharmaceutical preparations, such as tablets, pills, pulvis, powders, granules, troches, capsules, etc.; or liquid pharmaceutical preparations, such as solutions, suspensions, emulsions, syrups, elixirs, etc.

In manufacturing these pharmaceutical preparations, depending on the form of administration, it is possible to use general carriers such as excipients, diluents, binders, moisturizing agents, disintegrators, disintegration inhibitors, absorption accelerators, lubricants, solubilizers, buffers, emulsifiers, suspension agents, etc. Examples of additives include those that usually used depending on the form of administration, such as stabilizer, preservative, buffer, isotonizing agent, chelating agent, pH controller, surfactant, coloring agent, fragrances, flavoring agent, sweetening agent, etc.

The amount of carbohydrase inhibitor contained in the medical composition of the present invention depends on the form of the pharmaceutical preparation and cannot be defined unconditionally, but is usually selected from a range so that the final pharmaceutical preparation contains carbohydrase inhibitor in an amount of 0.001-100 wt %, and preferably 0.01-80 wt %.

The administration amount of the medical composition are not limited, and can be suitably selected from a wide range depending on the targeted treatment effects, administration method, treatment period, sex and or age of the subject, etc. The administration amount, e.g., the dose of carbohydrase inhibitor administered to a person whose body weight is 60 kg, depends on the administration route, and can be suitably selected from a range of about 10 to 200,000 mg/(60 kg body weight).

### EXAMPLES

The following Examples and Experiments are intended to illustrate the present invention in further detail, and not to limit the scope of the invention. In the Examples and Experiments, “%” means “% w/w” unless otherwise specified.

### Preparation Example 1 Chestnut Astringent Skin Extract

Dried chestnut astringent skin was pulverized to prepare a chestnut astringent skin powder. Two liters of 50% v/v aqueous acetonitrile solution was added to 100 g of the chestnut astringent skin powder, followed by stirring at room temperature for 5 hours. The mixture was then centrifuged at 3000 g for 15 minutes, and the resulting supernatant was concentrated in a rotary evaporator and lyophilized, to thereby obtain 6.8 g of an aqueous acetonitrile extract of chestnut astringent skin (lyophilized product).

### Preparation Example 2 Chestnut Shell Extract

Dried chestnut shells were pulverized to prepare a chestnut shell powder. One hundred grams of the chestnut shell powder was subjected to the same procedure as in Preparation Example 1, to thereby obtain 7.5 g of an aqueous acetonitrile extract of chestnut shells (lyophilized product).

### Preparation Example 3 Chestnut Leaf Extract

Dried chestnut leaves were pulverized to prepare a chestnut leaf powder. One hundred grams of the chestnut leaf powder was subjected to the same procedure as in Preparation Example 1, to thereby obtain 21.0 g of an aqueous acetonitrile extract of chestnut leaves (lyophilized product).

### Preparation Example 4 Chestnut Astringent Skin Extract

Dried chestnut astringent skin was pulverized to prepare a chestnut astringent skin powder. Two liters of 50% v/v aqueous acetone solution was added to 100 g of the chestnut astringent skin powder, followed by stirring at room temperature for 5 hours. The mixture was then centrifuged at 3000 g for 15 minutes, and the resulting supernatant was concentrated in a rotary evaporator and lyophilized to thereby obtain 6.5 g of an aqueous acetone extract of chestnut astringent skin (lyophilized product).

### Preparation Example 5 Chestnut Bark Extract

Dried chestnut bark was pulverized to prepare a chestnut bark powder. Two hundred milliliters of 50% v/v aqueous acetone solution was added to 10 g of the chestnut bark powder, followed by stirring at room temperature for 5 hours. The mixture was then centrifuged at 3000 g for 15 minutes, and the resulting supernatant was concentrated in a rotary evaporator and lyophilized, to thereby obtain 2.6 g of an aqueous acetone extract of chestnut bark (lyophilized product).

### Preparation Example 6 Chestnut Bur Spine Extract

Dried chestnut bur spines were pulverized to prepare a chestnut bur spine powder. Ten grams of the chestnut bur spine powder was subjected to the same procedure as in Preparation Example 5, to thereby obtain 1.9 g of an aqueous acetone extract of chestnut bur spines (lyophilized product).

### Preparation Example 7 Chestnut Bur Flesh Extract

Dried chestnut bur flesh were pulverized to prepare a chestnut bur flesh powder. Ten grams of the chestnut bur flesh powder was subjected to the same procedure as in Preparation Example 5, to thereby obtain 2.6 g of an aqueous acetone extract of chestnut bur flesh (lyophilized product).

### Preparation Example 8 Chestnut Seed (Nut and Cotyledon) Extract

Dried chestnut seeds (nut and cotyledons) were pulverized to prepare a chestnut seed powder. Ten grams of the chestnut seed powder was subjected to the same procedure as in Preparation Example 5, to thereby obtain 0.16 g of an aqueous acetone extract of chestnut seeds (nut and cotyledons) (lyophilized product).

### Preparation Example 9 Sawtooth Oak Nut Shell Extract

Dried sawtooth oak nut shells were pulverized to prepare a sawtooth oak nut shell powder. Ten grams of the sawtooth oak nut shell powder was subjected to the same procedure as in Preparation Example 5, to thereby obtain 0.64 g of an aqueous acetone extract of sawtooth oak nut shells (lyophilized product).

### Preparation Example 10 Sawtooth Oak Seed (Nut and Cotyledon) Extract

Dried sawtooth oak seeds (nut and cotyledons) were pulverized to prepare a sawtooth oak seed powder.

Ten grams of the sawtooth oak seed powder was subjected to the same procedure as in Preparation Example 5, to thereby obtain 1.92 g of an aqueous acetone extract of sawtooth oak seeds (nut and cotyledons) (lyophilized product).

### Preparation Example 11 Suda-jii (Castanopsis sieboldii) Nut Shell Extract

Dried Suda-jii nut shells were pulverized to prepare a Suda-jii nut shell powder. Ten grams of the Suda-jii nut shell powder was subjected to the same procedure as in Preparation Example 5, to thereby obtain 0.68 g of an aqueous acetone extract of Suda-jii nut shells (lyophilized product).

### Preparation Example 12 Suda-jii Seed (Nut and Cotyledon) Extract

Dried Suda-jii seeds (nut and cotyledons) were pulverized to prepare a Suda-jii seed powder. Ten grams of the Suda-jii seed powder was subjected to the same procedure as in Preparation Example 5, to thereby obtain 0.85 g of an aqueous acetone extract of Suda-jii seeds (lyophilized product).

### Comparative Preparation Example Hot Water Extract of Guava Leaves

Dried guava leaves were pulverized to prepare a guava leaf powder. Two liters of water was added to 100 g of the guava leaf powder, followed by stirring at 100° C. for 1 hour. The mixture was then centrifuged at 3000 g for 15 minutes, and the resulting supernatant was concentrated in a rotary evaporator and lyophilized, to thereby obtain 17.6 g of a hot water extract of guava leaves.

Experiment 1 α-Amylase Inhibitory Activity Test

(1) The chestnut astringent skin extract, chestnut shell extract and chestnut leaf extract of Preparation Examples 1 to 3, and the hot water extract of guava leaves of the Comparative Preparation Example were tested for inhibitory activity against α-amylase. Each of the above extracts was dissolved in 200 mM phosphate buffer (pH 7.0) to final concentrations of 2.7, 5.5, 8.2, 22, 55 and 110 μg/ml to use as test inhibitors, in this test.

Specifically, 1.0 ml of buffer (200 mM phosphate buffer, pH 7.0), 0.5 ml of 1% aqueous sodium chloride solution, 2.5 ml of 0.25% soluble starch solution in 200 mM phosphate buffer at pH 7.0, and 0.5 ml of one of the test inhibitors at one of the above concentrations were mixed together, and pig pancreatic α-amylase (Sigma) was added in an amount of 50 μl (about 1.6 U; 1 U being the amount required to release 1 mg of maltose from starch per 3 minutes at 20° C. and pH 6.8), followed by a reaction at 37° C. for 30 minutes. Subsequently, 0.5 ml of 8% aqueous sodium hydroxide was added to the reaction mixture to terminate the reaction, and 0.5 ml dinitrosalicylic acid reagent [prepared by mixing 50 ml of potassium sodium tartrate solution (30 g/50 ml of purified water) and 20 ml of 3,5-dinitrosalicylic acid solution (1 g/20 ml of 8% aqueous sodium hydroxide) and diluting the mixture with purified water to 100 ml] was added. The resulting mixture was heated at 100° C. for 5 minutes and then cooled, and its absorbance at 540 nm was measured. The measured absorbance is referred to as B. As a blank test, the above procedure was repeated using 50 μl of purified water in place of 50 μl pig pancreatic α-amylase, and the absorbance at 540 nm was measured. This measured absorbance is referred to as D. Further, the above procedure was repeated using 0.5 ml of purified water in place of 0.5 ml of the test inhibitor, and the absorbance at 540 nm was measured. This measured absorbance is referred to as A. Furthermore, the above procedure was repeated using 0.55 ml of purified water in place of 0.5 ml of the test inhibitor and 50 μl of α-amylase, and the absorbance at 540 nm was measured. This measured absorbance is referred to as C.

The α-amylase activity (%) in each reaction was calculated from the absorbances A, B, C and D by the following equation:
α-Amylase activity (%)={(B−D)/(A−C)}×100

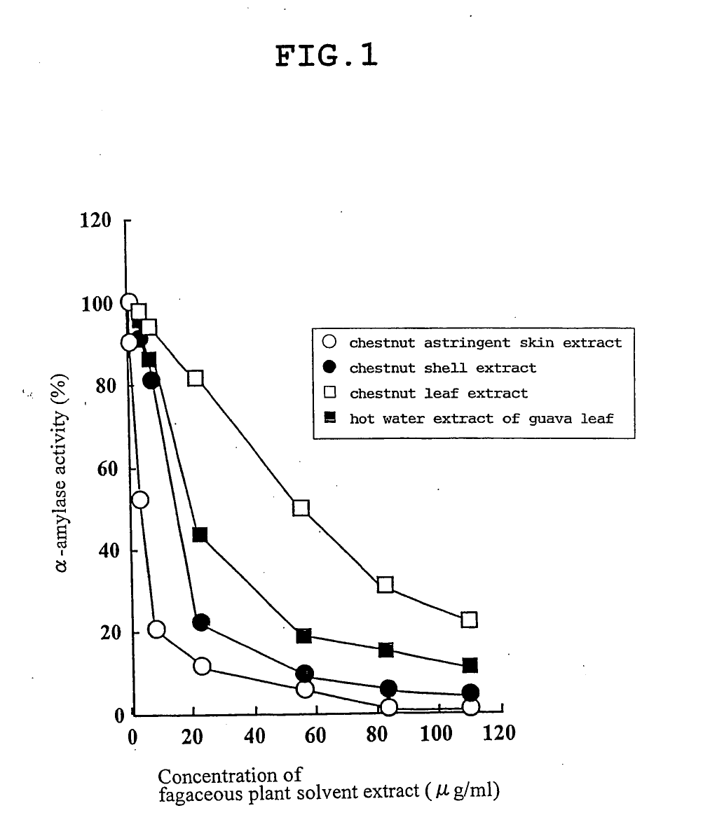

FIG. 1 shows the α-amylase activity (%) of each reaction system, plotting the concentration (μg/ml) of each extract used as a test inhibitor as abscissa. FIG. 1 reveals that all of the chestnut astringent skin extract (-◯-), chestnut shell extract (-●-) and chestnut leaf extract (-□-) inhibited α-amylase activity in a concentration dependent manner, demonstrating that these extracts have α-amylase inhibitory activity. The chestnut astringent skin extract and chestnut shell extract exhibited higher α-amylase inhibitory activity than the hot water extract of guava leaves, which is known to have α-amylase inhibitory activity (e.g., from Japanese Unexamined Patent Publication No. 1995-59539).

(2) The α-amylase inhibitory activities of the chestnut bark extract, chestnut bur spine extract, chestnut bur flesh extract, chestnut seed (nut and cotyledon) extract, sawtooth oak nut shell extract, sawtooth oak seed (nut and cotyledon) extract, Suda-jii nut shell extract, and Suda-jii seed (nut and cotyledon) extract, prepared in Preparation Examples 5 to 12, were tested according to the method described above in (1).

The 50% inhibitory concentrations of these extracts were calculated from the inhibitory activities. Table 1 shows the results and the 50% inhibitory concentrations of the chestnut astringent skin extract (Preparation Example 1), chestnut shell extract (Preparation Example 2), chestnut leaf extract (Preparation Example 3) and hot water extract of guava leaves (Comparative Preparation Example) measured above in (1).

TABLE 1
Fifty percentage inhibitory concentrations of the extracts
for α-amylase

| Prep. | | 50% Inhibitory | |
|---|---|---|---|
| Ex. | Extract | concentration | |
| 1 | Chestnut astringent skin extract | 3.1 | μg/ml |
| 2 | Chestnut shell extract | 15.2 | μg/ml |
| 3 | Chestnut leaf extract | 56.1 | μg/ml |
| 5 | Chestnut bark extract | 54.9 | μg/ml |
| 6 | Chestnut bur spine extract | 257.0 | μg/ml |
| 7 | Chestnut bur flesh extract | 447.0 | μg/ml |
| 8 | Chestnut seed (nut and | 1 | mg/ml or more |
| | cotyledon) extract | | |
| 9 | Sawtooth nut shell extract | 4.4 | μg/ml |
| 10 | Sawtooth seed (nut and | 629.0 | μg/ml |
| | cotyledon) extract | | |
| 11 | Suda-jii nut shell extract | 12.4 | μg/ml |
| 12 | Suda-jii seed (nut and | 1 | mg/ml or more |
| | cotyledon) extract | | |
| Comp. | Guava leaf extract | 25.0 | μg/ml |

As is apparent from these results, among these fagaceous plant extracts, the chestnut astringent skin extract and sawtooth oak nut shell extract have a low 50% inhibitory concentration against α-amylase of about one-sixth to about one-eighth of that of the hot water extract of guava leaves, demonstrating that they have extremely high α-amylase inhibitory activity. The extracts of sawtooth oak nut shells and Suda-jii nut shells have a low 50% inhibitory concentration of about two-thirds to about one-half of that of the hot water extract of guava leaves, showing their high α-amylase inhibitory activity. The extracts of leaves, bark and burs (spines and flesh) of chestnut also exhibited α-amylase activity.

With respect to seed (nut and cotyledon) extracts, the sawtooth oak seed extract exhibited significant α-amylase inhibitory activity, but chestnut seed and Suda-jii seed extracts showed extremely low α-amylase inhibitory activity.

Experiment 2 α-glucosidase Inhibitory Activity Test

The chestnut astringent skin extract (Preparation Example 1), chestnut shell extract (Preparation Example 2), chestnut leaf extract (Preparation Example 3), chestnut bark extract (Preparation Example 5), chestnut bur spine extract (Preparation Example 6), chestnut bur flesh extract (Preparation Example 7), chestnut seed (nut and cotyledon) extract (Preparation Example 8), sawtooth oak nut shell extract (Preparation Example 9), sawtooth oak seed (nut and cotyledon) extract (Preparation Example 10), Suda-jii nut shell extract (Preparation Example 11), and Suda-jii seed (nut and cotyledon) extract (Preparation Example 12) and hot water extract of guava leaves (Comparative Preparation Example) were Tested for Inhibitory Activity against α-glucosidases (maltase and sucrase). Each of the above extracts was dissolved in 80 mM phosphate buffer (pH 7.0) to final concentrations of 0.13, 0.25, 0.5 and 1.0 mg/ml to use as test inhibitors in this test.

(1) Preparation of α-glucosidase Solution

An α-glucosidase solution was prepared according to Anal. Biochem 7: 18-25, 1964. Specifically, small intestines were excised from rats, washed with physiological saline, and everted. The jejunal mucosal cells were scraped with a glass slide, placed in a Teflon® homogenizer containing 80 mM phosphate buffer (pH 7.0) and homogenized on ice. The phosphate buffer was used in an amount of 40 ml with respect to jejunal mucosal cells obtained from 4 rats. The homogenized cells were centrifuged (1000 g, 10 min, 4° C.), and the supernatant was used as an α-glucosidase solution.

(2) Measurement of Maltase Inhibitory Activity

(2-1) Fifty microliters of α-glucosidase solution prepared above in (1) was added to a mixture of 400 μl of 50 mM maltose solution (substrate solution) in phosphate buffer and 50 μl of one of the test inhibitors, and the resulting mixture was maintained at 37° C. for 30 minutes. Subsequently, the reaction was terminated in a boiling water bath for 2 minutes, and the reaction mixture was then ice-cooled. The glucose released in the reaction mixture was measured by a glucose measurement kit (Glucose C-II Test Wako, Wako Pure Chemical Ind. Ltd.). The measured amount of glucose is referred to as B. As a blank test, the above procedure was repeated using 50 μl of purified water in place of 50 μl of the α-glucosidase solution, and the amount of released glucose was measured. This measured amount of glucose is referred to as D. Further, the above procedure was repeated using 50 μl of purified water in place of 50 μl of the test inhibitor, and the amount of released glucose was measured. This measured amount of glucose is referred to as A. Furthermore, the above procedure was repeated using 100 μl of purified water in place of 50 μl of the test inhibitor and 50 μl of the α-glucosidase solution, and the amount of released glucose was measured. This measured amount of glucose is referred to as C.

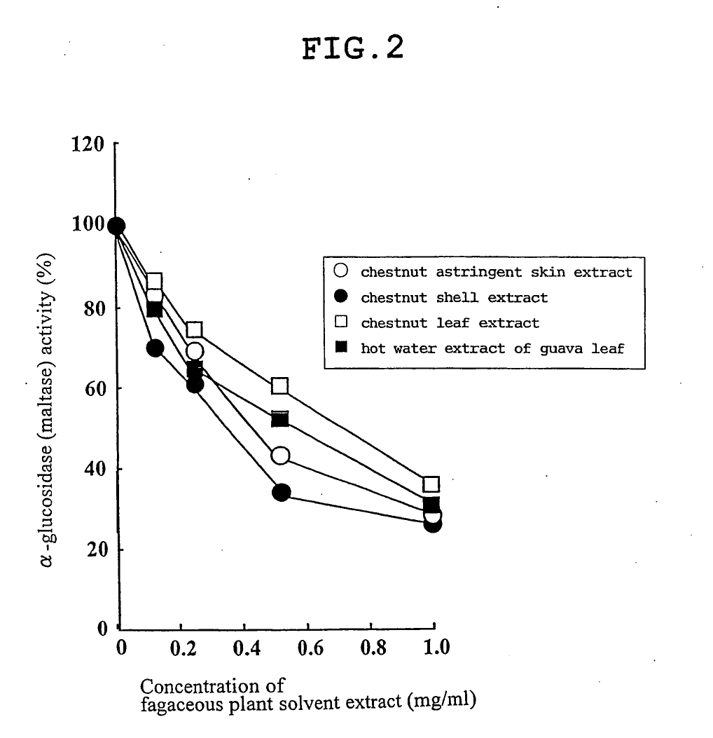

The maltase activity (%) in each reaction system was calculated from the glucose amounts A, B, C and D by the following equation:
Maltase activity (%)={(B−D)/(A−C)}×100
(2-2) FIG. 2 shows the maltase activity (%) of each reaction, plotting the concentration (mg/ml) of each extract used as a test inhibitor [chestnut astringent skin extract (Preparation Example 1), chestnut shell extract (Preparation Example 2), chestnut leaf extract (Preparation Example 3), or hot water extract of guava leaves (Comparative Preparation Example)] as abscissa. FIG. 2 reveals that all of the chestnut astringent skin extract (-◯-), chestnut shell extract (-●-) and chestnut leaf extract (-□-) inhibited maltose activity in a concentration dependent manner, demonstrating that these extracts have α-glucosidase (maltase) inhibitory activity. The chestnut astringent skin extract and chestnut shell extract exhibited α-glucosidase (maltase) inhibitory activity equivalent to or higher than that of the hot water extract of guava leaves, which is known to have α-glucosidase (maltase) inhibitory activity (“Food Science & Business”, offprint from Nikkei Biotechnology & Business, pp. 108-111, 2003).

(2-3) Table 2 shows the 50% inhibitory concentrations (mg/ml) of the extracts prepared in Preparation Examples 1 to 3 and 5 to 12 and the Comparative Preparation Example against maltase activity.

TABLE 2
Fifty percentage inhibitory concentrations of the extracts
for maltase

| Prep. | | 50% Inhibitory | |
|---|---|---|---|
| Ex. | Extract | concentration | |
| 1 | Chestnut astringent skin extract | 0.430 | mg/ml |
| 2 | Chestnut shell extract | 0.351 | mg/ml |
| 3 | Chestnut leaf extract | 0.706 | mg/ml |
| 5 | Chestnut bark extract | 0.241 | mg/ml |
| 6 | Chestnut bur spine extract | 0.365 | mg/ml |
| 7 | Chestnut bur flesh extract | 0.431 | mg/ml |
| 8 | Chestnut seed (nut and cotyledon) | 1 | mg/ml or more |
| | extract | | |
| 9 | Sawtooth nut shell extract | 0.727 | mg/ml |
| 10 | Sawtooth seed (nut and cotyledon) | 0.919 | mg/ml |
| | extract | | |
| 11 | Suda-jii nut shell extract | 0.870 | mg/ml |
| 12 | Suda-jii seed (nut and cotyledon) | 1 | mg/ml or more |
| | extract | | |
| Comp. | Guava leaf extract | 0.557 | mg/ml |

Table 2 reveals that, like the chestnut astringent skin extract and chestnut shell extract, the chestnut bark extract and chestnut bur (spines and flesh) extracts showed α-glucosidase (maltase) inhibitory activity equivalent to or better than that of the hot water extract of guava leaves. The extracts of sawtooth oak nut shells and Suda-jii nut shells exhibited maltase inhibitory activity, which is, however, worse than that of the chestnut shell extract and chestnut astringent skin extract. With respect to seed (nut and cotyledon) extracts, the sawtooth seed extract showed maltase inhibitory activity, but the chestnut seed and Suda-jii seed (nut and cotyledon) extracts exhibit extremely poor inhibitory activity.

(3) Measurement of Sucrase Inhibitory Activity

(3-1) The procedure described above in (2-1) was repeated using, as a substrate solution, 400 μl of 50 mM sucrose solution in phosphate buffer in place of 400 μl of 50 mM maltose solution in phosphate buffer, and the amount of released glucose in the reaction mixture was measured.

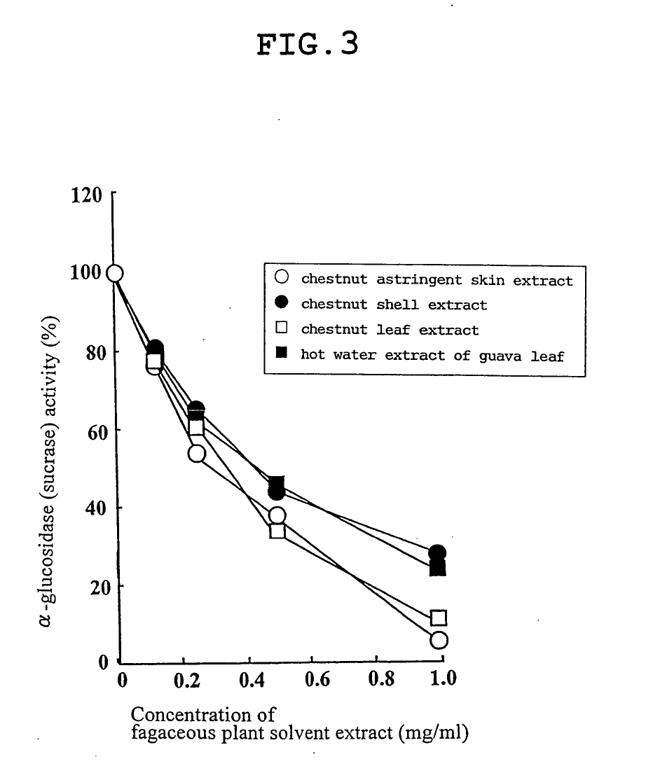

The sucrase activity (%) in each reaction was calculated from the glucose amounts A, B, C and D by the following equation:
Sucrase (%)={(B−D)/(A−C)}×100
(3-2) FIG. 3 shows the sucrase activity (%) of each reaction, plotting the concentration of each extract used as a test inhibitor [the chestnut astringent skin extract (Preparation Example 1), chestnut shell extract (Preparation Example 2), chestnut leaf extract (Preparation Example 3), or hot water extract of guava leaves (Comparative Preparation Example 1)] as abscissa. FIG. 3 reveals that all of the chestnut astringent skin extract (-◯-), chestnut shell extract (-●-) and chestnut leaf extract (-□-) inhibited sucrase activity concentration-dependently, demonstrating that these extracts have α-glucosidase (sucrase) inhibitory activity. The chestnut astringent skin extract, chestnut shell extract and chestnut leaf extract exhibited α-glucosidase (sucrase) inhibitory activity equivalent to or higher than that of the hot extract of guava leaves, which is known to have α-glucosidase (sucrase) inhibitory activity (“Food Science & Business”, offprint from Nikkei Biotechnology & Business, pp. 108-111, 2003).

(3-3) Table 3 shows the 50% inhibitory concentrations (mg/ml) of the extracts prepared in Preparation Examples 1 to 3 and 5 to 12 and the Comparative Preparation Example against sucrase activity.

TABLE 3
Fifty percentage inhibitory concentrations of the extracts
for sucrase.

| Prep. | | 50% Inhibitory | |
|---|---|---|---|
| Ex. | Extract | concentration | |
| 1 | Chestnut astringent skin extract | 0.352 | mg/ml |
| 2 | Chestnut shell extract | 0.341 | mg/ml |
| 3 | Chestnut leaf extract | 0.300 | mg/ml |
| 5 | Chestnut bark extract | 0.449 | mg/ml |
| 6 | Chestnut bur spine extract | 0.433 | mg/ml |
| 7 | Chestnut bur flesh extract | 0.288 | mg/ml |
| 8 | Chestnut seed (nut and cotyledon) | 1 | mg/ml or more |
| | extract | | |
| 9 | Sawtooth nut shell extract | 0.869 | mg/ml |
| 10 | Sawtooth seed (nut and cotyledon) | 0.310 | mg/ml |
| | extract | | |
| 11 | Suda-jii nut shell extract | 1 | mg/ml or more |
| 12 | Suda-jii seed (nut and cotyledon) | 1 | mg/ml or more |
| | extract | | |
| Comp. | Guava leaf extract | 0.433 | mg/ml |

Table 3 reveals that, like the chestnut astringent skin extract and chestnut shell extract, the chestnut leaf extract, chestnut bark extract, chestnut bur spine extract, chestnut bur flesh extract and sawtooth oak seed (nut and cotyledon) extract exhibited α-glucosidase (sucrase) inhibitory activity equivalent to or higher than that of the hot water extract of guava leaves. The sawtooth nut shell showed sucrase inhibitory activity, which is, however, lower than that of the chestnut shell extract and chestnut astringent skin extract. The Suda-jii nut shell extract, chestnut seed (nut and cotyledon extract and Suda-jii seed (nut and cotyledon) extract, showed extremely poor α-glucosidase (sucrase) inhibitory activity.

Experiment 3 Carbohydrate Tolerance Test on Normal Rats

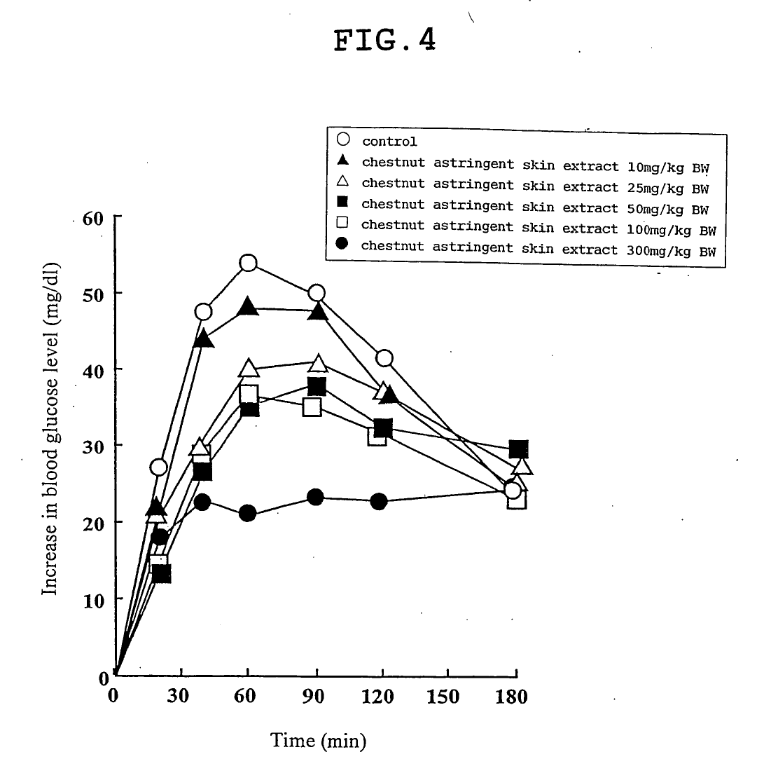

Male Wister rats weighing 150 g (Japan Clea Inc.) were preliminarily fed for 1 week, and those then weighing 180 g to 230 g were subjected to the following carbohydrate tolerance test (n=8 to 10 per group). Specifically, the rats were fasted for 12 hours, and the chestnut astringent skin extract (10 mg/kg body weight -▴-; 25 mg/kg body weight -Δ-; 50 mg/kg body weight -▪-; 100 mg/kg body weight -□-; and 300 mg/kg body weight -●-) prepared in Preparation Example 1 and starch (2 g/kg body weight) were administered at the same time to the rats through a gastric tube. Blood samples were collected from the caudal vessel 0, 20, 40, 60, 90, 120 and 180 minutes after the administration, and the blood glucose levels (mg/dl) of the samples were measured to examine the changes in blood glucose levels, using a Glucocard (DIAmeter-α, ARKRAY, INC). As a control test, rats were fasted for 12 hours and given only starch (2 g/kg body weight), without the chestnut astringent skin extract, through a gastric tube, and the blood glucose levels (mg/dl) were measured at the same time points and under the same conditions as above (-◯-). FIG. 4 shows the results. The ordinate of FIG. 4 represents the increase in blood glucose levels (mg/dl) over the blood glucose levels before the administration of the test substances.

In the control rats, the blood glucose level rapidly increased until 60 minutes after the administration, whereas in the rats given the chestnut astringent skin extract, the increase in blood glucose level was remarkably suppressed depending on the concentration of the chestnut astringent skin extract administered. This is presumably because the chestnut astringent skin extract inhibits the activities of α-amylase and α-glucosidase in the body, thereby slowing down glycolysis and suppressing carbohydrate absorption.

Experiment 4 Carbohydrate Tolerance Test on Diabetic Model Rats

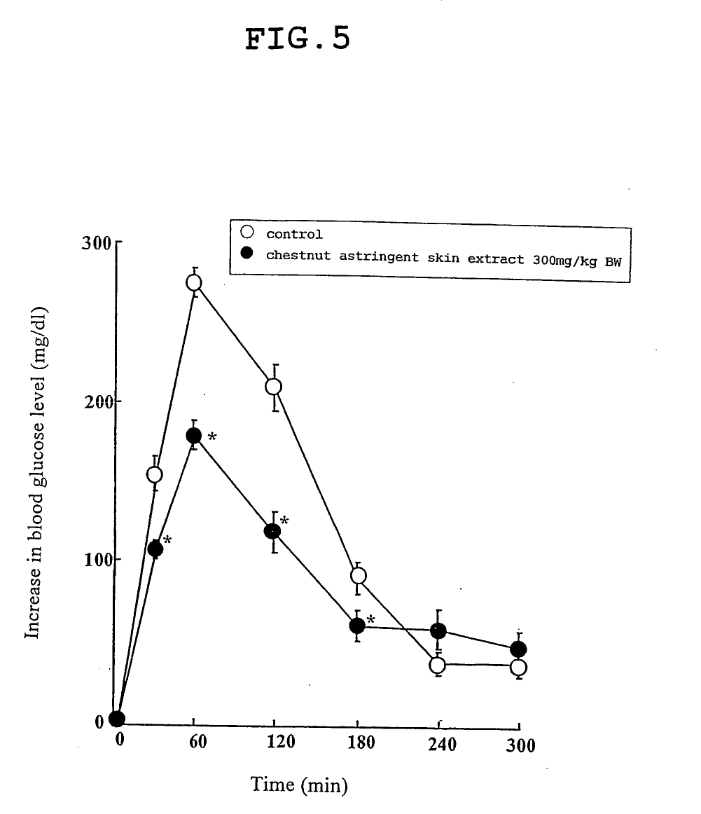

Male type II diabetic model rats (GK/jcl, Japan Clea Inc.) weighing 250 g were preliminarily fed for 1 week and subjected to the following carbohydrate tolerance test (n=8 per group). Specifically, the rats were fasted for 12 hours, and the chestnut astringent skin extract prepared in Preparation Example 1 (300 mg/kg body weight) and starch (2 g/kg body weight) were administered to the rats through a gastric tube (-●-). Blood samples were collected from the caudal vessel 0, 20, 30, 60, 120, 180, 240 and 300 minutes after the administration, and the blood glucose levels (mg/dl) of the samples were measured to examine the changes in blood glucose levels, using a Glucocard (DIAmeter-α, ARKRAY, INC). As a control test, rats were fasted for 12 hours and given only starch (2 g/kg body weight), without the chestnut astringent skin extract, through a gastric tube, and the blood glucose levels were measured over time and under the same conditions as above. One week after the administration period, the rats in the control group and rats in the chestnut astringent skin extract administration group were swapped with each other, and the above test procedure was repeated. FIG. 5 shows the results. The ordinate of FIG. 5 represents the increase in blood glucose levels (mg/dl) over the blood glucose levels before the administration. In FIG. 5, the “Increase in blood glucose level” is the mean of the values before and after the swapping of the rats.

The diabetic model rats had a high blood glucose of 100 mg/dl even after 12 hours of fasting. As is apparent from FIG. 5, in the control group, the blood-sugar level rapidly increased until 60 minutes due to the administration of the starch, whereas in the chestnut astringent skin extract administration group, this increase was significantly suppressed. This suggests that, even in rats with diabetes, the chestnut astringent skin extract inhibits the activities of α-amylase and α-glucosidase in the body, thereby slowing down glycolysis and suppressing carbohydrate absorption.

Experiment 5 Carbohydrate Tolerance Test on Humans

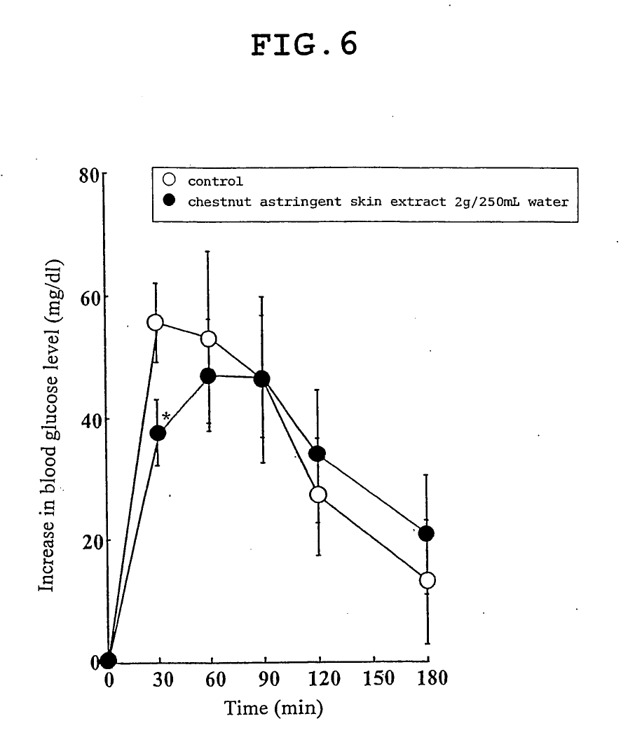

A carbohydrate tolerance test were performed on 11 volunteers (25 to 63 years old), according to Helsinki Declaration. The volunteers were fasted for 11.5 hours, i.e., from 9:00 p.m. the night before to initiation of the experiment. After measuring blood glucose levels (fasting blood levels) at 8:30 a.m., the volunteers ingested 200 g of boiled rice. At the time of the ingestion of boiled rice, five of the 11 volunteers ingested 2 g of chestnut astringent skin extract (Preparation Example 1) in 250 ml of water (chestnut astringent skin extract ingestion group), and the remaining six ingested 250 ml of water only (control group). blood glucose levels (mg/dl) were measured 30, 60, 90, 120 and 180 minutes after the ingestion, using a Glucbcard (DIAmeter-α, ARKRAY, INC). One week thereafter, the volunteers in the chestnut astringent skin extract ingestion group and those in the control group were swapped with each other, and the same test procedure was repeated. FIG. 6 shows the results. The ordinate of FIG. 6 represents the increase in blood-sugar levels (mg/dl) over the blood-sugar levels before the ingestion of boiled rice.

When only water was ingested together with boiled rice, the blood glucose levels rapidly increased until 30 minutes after the ingestion (control group: -◯-). In contrast, when water and the chestnut astringent skin extract were ingested together with boiled rice, this increase in blood glucose level was significantly suppressed (chestnut astringent skin extract ingestion group: -●-).

From 120 minutes after the ingestion, the increase in blood glucose level in the chestnut astringent skin extract ingestion group was slightly greater than that in the control group. This is presumably because the chestnut astringent skin extract inhibits the activities of α-amylase and α-glucosidase in the body, thereby slowing down glycolysis and suppressing carbohydrate absorption.

The above results demonstrate that the chestnut astringent skin extract of the present invention is effective for suppressing an increase in blood glucose levels (alleviating hyperglycemia) in humans.

### Example 1 Noodles

Noodles were prepared from 500 g of medium-strength flour, 30 g of salt, 500 mg of the aqueous acetone extract of chestnut astringent skin obtained in Preparation Example 4, and 200 g of water.

### Example 2 Hamburger Patty

A hamburger patty was prepared from 22.5 g of minced beef, 20.0 g of minced pork, 20.0 g of onion, 7.5 g of bread crumbs, 23 g of water, 2 g of salt, 1 g of sugar, 1 g of spice, 2 g of purified rapeseed oil, and 1 g of the aqueous acetone extract of chestnut astringent skin extract obtained in Preparation Example 4.

### Example 3 Soft Drink

Hot water (1000 ml) was added to 10 g of black tea leaves to obtain an extract. One hundred grams of honey, 50 g of lemon juice, and 1 g of the aqueous acetone extract of chestnut astringent skin obtained in Preparation Example 4 were added to the extract, to thereby obtain a soft drink.

Although the aqueous acetone extract of chestnut astringent skin extract obtained in Preparation Example 4 was used as a carbohydrase inhibitor in Examples 1 to 3, any one of the fagaceous plant solvent extracts obtained in Preparation Examples 1 to 3, and 5 to 11 can be used in place of the extract of Preparation Example 4 to prepare noodles, hamburger patties or soft drinks.

### INDUSTRIAL APPLICABILITY

A substance having the carbohydrase inhibitory activity of the present invention (carbohydrase inhibitor) has an excellent inhibitory activity against α-amylase or α-glucosidase. Among such substances, it is clear that a carbohydrase inhibitor derived from an astringent skin or the like of chestnut is safe for living bodies based on years of dietary experience.

Therefore, the carbohydrase inhibitor of the present invention is effective for reducing or preventing obesity by suppressing digestion and absorption of saccharides from alimentary canal. Furthermore, the carbohydrase inhibitor of the present invention can delay saccharide digestion and absorption and suppress a rise in postprandial blood glucose levels, and therefore it can be effectively used for ameliorating diabetic hyperglycemic conditions and preventing development in disorders of a diabetic patient caused by hyperglycemia.

Furthermore, a food composition comprising the carbohydrase inhibitor of the present invention is expected to prevent the development of diseases attributable to obesity caused by hyperphagia by the ability to inhibit digestion of starches and sugars contained in foods and preventing them being converted to energy. Furthermore, since the food composition of the present invention can suppress a rise in postprandial blood glucose levels by delaying digestion and absorption of saccharides, the food composition of the present invention is expected to have an effect in preventing or ameliorating diabetes. For example, by mixing the carbohydrase inhibitor of the present invention with foods containing a lot of starch, it is possible to provide foods for those who have high blood glucose levels or those who would like to reduce their obesity.

In recent years, production of peeled chestnuts has been increasing, and a large volume of chestnut testa is disposed as industrial waste. According to the present invention, such chestnut testae (astringent skin and shell) can be effectively utilized. In particular, chestnut astringent skins contained in roast chesnuts and marron glacé are edible, and therefore there is no reason to doubt its safety in living bodies.

1. An α-amylase inhibitor comprising a fagaceous plant solvent extract obtained by subjecting the astringent skin of a plant of the Castanea genus, the shell of Castanopsis sieboldii, or the shell of Quercus acutissima to extraction using water, organic solvent, or a mixture thereof.

2. (canceled)

3. (canceled)

4. The α-amylase inhibitor according to claim 1, wherein the plant of the Castanea genus is Castanea crenata.

5. (canceled)

6. A food composition comprising an α-amylase inhibitor of claim 1.

7. The food composition according to claim 6, which is a beverage or a food high in carbohydrates.

8. (canceled)

9. The food composition according to claim 6, which comprises an effective amount of an α-amylase inhibitor of claims 1 as an active ingredient for delaying saccharide digestion and absorption.

10. The food composition according to claim 9, which has a property of delaying saccharide digestion and absorption, and whose package has a note stating that the food composition is suitable for delaying saccharide digestion and absorption.

11. The food composition according to claim 6, which comprises an effective amount of an α-amylase inhibitor of claim 1 as an active ingredient for reducing a postprandial rise in blood glucose

12. The food composition according to claim 11, which has a property of suppressing postprandial rise in blood glucose levels or for ameliorating hyperglycemia, and whose package has a note stating that the food composition is suitable for suppressing a postprandial rise in blood glucose levels or for ameliorating hyperglycemia.

13. The food composition according to claim 6, which comprises an α-amylase inhibitor of claim 1 as an active ingredient in an amount effective for preventing obesity.

14. The food composition according to claim 13, which has an anti-obesity effect, and whose package has a note stating that the food composition is suitable for preventing obesity.

15. A medical composition comprising an α-amylase inhibitor of claim 1 as an active ingredient, together with a pharmaceutically acceptable carrier(s) and/or additive(s).

16. The medical composition according to claim 15, which is a medicine for preventing or treating diabetes.

17. The medical composition according to claim 15, which is an anti-obesity medicine.

18. A method for suppressing a postprandial rise in blood glucose levels or ameliorating hyperglycemia of a subject, comprising injecting or otherwise administering an α-amylase inhibitor of claim 1 to the subject.

19. A method for preventing or reducing obesity comprising injecting or otherwise administering an α-amylase inhibitor of claim 1 to the subject.

20. Use of an α-amylase inhibitor of claim 1 for production of a composition for delaying saccharide digestion and absorption, a composition for suppressing a postprandial rise in blood glucose levels, a composition for ameliorating hyperglycemia, or an anti-obesity composition.
